(19) United States
(12) Patent Application Publication
(10) Pub. No.: US 2009/0099365 A1
(43) Pub. Date: Apr. 16, 2009

(54) Processes for solifenacin preparation

(76) Inventors: Nurit Perlman, Kfar Saba (IL); Gideon Pilarski, Holon (IL)

Correspondence Address: KENYON & KENYON LLP, ONE BROADWAY, NEW YORK, NY, 10004, US

(21) Appl. No.: 12/218,401

(22) Filed: Jul. 14, 2008

Related U.S. Application Data
(60) Provisional application No. 60/949,721, filed on Jul. 13, 2007.
(60) Provisional application No. 61/030,145, filed on Feb. 20, 2008.
(60) Provisional application No. 61/050,885, filed on May 6, 2008.

(51) Int. Cl. C07D 453/04

(52) U.S. Cl. 546/134

(57) ABSTRACT

Processes for preparing solifenacin comprising distilling ethanol and organic solvent from a reaction mixture and recycling the organic solvent are described. Also described are processes for reducing solifenacin diastereomeric and enantiomeric impurities.

### CROSS-REFERENCE TO RELATED APPLICATIONS

This application claims the benefit of Provisional Application Ser. No. 60/949,721, filed Jul. 13, 2007, Provisional Application Ser. No. 61/030,145, filed Feb. 20, 2008, and Provisional Application Ser. No. 61/050,885, filed May 6, 2008, each of which is incorporated herein by reference in its entirety.

### FIELD OF THE INVENTION

The present invention relates to processes for the preparation of solifenacin and salts thereof.

### BACKGROUND OF THE INVENTION

(3R)-1-azabicyclo[2.2.2]oct-3-yl-(1S)-1-phenyl-3,4-dihydroisoquinoline-2-(1H)-carboxylate or (S)-phenyl-1,2,3,4-tetrahydroisoquinoline-2-carboxylic acid 3(R)-quinuclidinyl ester having the following formula:

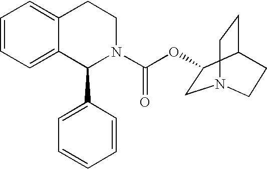

is known as solifenacin, also known as YM-905 (in its free base form) and YM-67905 (in its succinate form). Solifenacin has the molecular formula C₂₃H₂₆O₂, and a molecular weight of 362.4647.

One of the solifenacin salts is solifenacin succinate, which is a urinary antispasmodic, acting as a selective antagonist to the M(3)-receptor. It is used for treatment of symptoms of overactive bladder (“OAB”), such as urinary urgency and increased urinary frequency, as may occur in patients with overactive bladder syndrome, as reviewed in Chilman-Blair et al., Solifenacin: Treatment of overactive bladder, Drugs of Today, 40(4): 343-353 (2004).

The commercial solifenacin tablet is marketed under the trade name VESIcare®. VESIcare® was approved by the FDA for once daily treatment of OAB and is prescribed as 5 mg and 10 mg tablets.

U.S. Pat. No. 6,017,927 and its continuation, U.S. Pat. No. 6,174,896, purportedly describe compounds having the general formula:

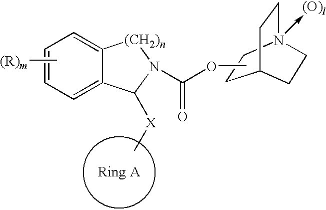

which includes solifenacin. Processes for the synthesis and pharmaceutical compositions containing solifenacin are also described, wherein solifenacin is obtained by admixing quinuclidinyl chloroformate monohydrochloride with (1R)-1-phenyl-1,2,3,4-tetrahydroisoquinoline, as described by the following scheme:

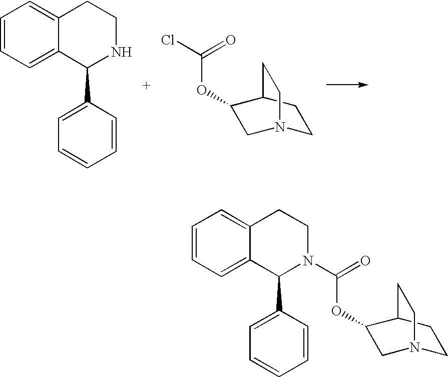

Mealy, N., et al. in Drugs of the Future, 24 (8): 871-874 (1999) (“Mealy”), which is incorporated herein by reference, purportedly describes transesterifying racemic 1-pheny-1,2,3,4-tetrahydroisoquinoline-2-carboxylic acid ethyl ester with quinuclidine-3(R)-ol by means of NaH in refluxing toluene to provide the quinuclidinyl ester as a diastereomeric mixture, illustrated by the following scheme:

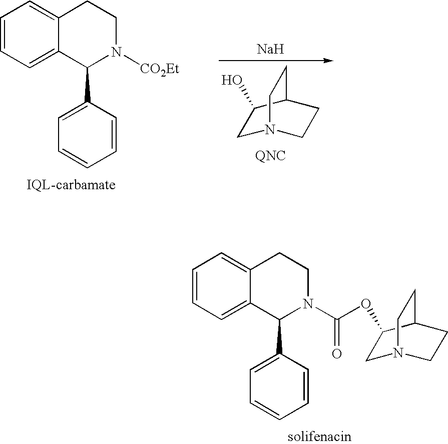

EP patent publication No. 1,726,304 (“the EP '304 publication”), which is incorporated herein by reference, purportedly describes a process for the preparation of solifenacin, shown by the following scheme:

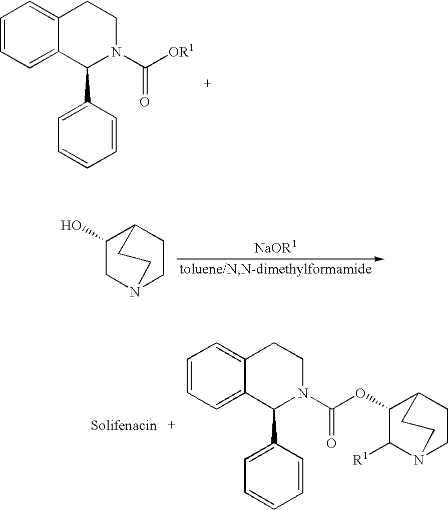

in the presence of alkali metal lower alkoxide, wherein R¹ represents optionally substituted lower alkyl.

EP patent publication No. 1,757,604 purportedly discloses processes for the production of solifenacin, shown by the following scheme:

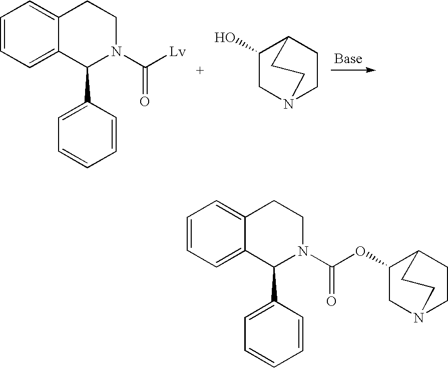

in the presence of alkali metal lower alkoxides, wherein Lv represents 1H-imidazol-1-yl, 2,5-dioxopyrrolidin-1-yloxy, 3-methyl-1H-imidazol-3-ium-1-yl or chloro.

EP patent publication No. 1,714,965 (“the EP '965 publication”), which corresponds to PCT publication no. WO 2005/075474, both of which are incorporated herein by reference, purportedly describes several solifenacin impurities and the importance of removing them from the obtained product in order to produce a pharmaceutical composition. Both the EP '304 and '965 publications discloses using an organic solvent to S-IQL-ethyl carbamate ratio of about 10:1 L/kg.

There is a need in the art for processes for preparing solifenacin and for preparing solifenacin with high purity.

### BRIEF DESCRIPTION OF THE FIGURES

FIG. 1 shows a Dean-Stark apparatus.

### SUMMARY OF THE INVENTION

In one embodiment, the present invention encompasses substantially pure solifenacin succinate. The substantially pure solifenacin succinate has less than about 0.20% of any single chemical impurity as measured by area under HPLC peak relative to the total area under all peaks. Preferably, the chemical purity of the solifenacin succinate is about 99% or more.

In one embodiment, the present invention encompasses a process for the production of solifenacin having the following formula:

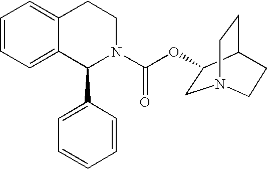

comprising combining (S)-1,2,3,4-tetrahydro-1-phenylisoquinoline-2-carboxylic acid ethyl ester (“S-IQL-ethyl carbamate”) of the following formula:

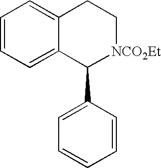

with (R)-1-azabicyclo[2.2.2]octan-3-ol (“QNC”) having the following formula:

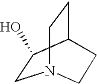

, and a solvent to obtain a mixture, wherein the ratio between the solvent and the S-IQL-ethyl carbamate is about 1:1 to about 2:1 ml/g.

In one embodiment, the present invention encompasses a process for the production of solifenacin having the following formula:

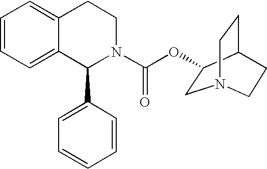

comprising combining (S)-1,2,3,4-tetrahydro-1-phenylisoquinoline-2-carboxylic acid ethyl ester (“S-IQL-ethyl carbamate”) of the following formula:

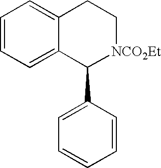

with (R)-1-azabicyclo[2.2.2]octan-3-ol (“QNC”) having the following formula:

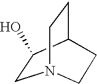

, a base, and an organic solvent to obtain a mixture; removing ethanol generated by azeotropic distillation with the organic solvent; and optionally recovering solifenacin. Preferably, the organic solvent removed is recycled. Preferably, the ratio between the solvent and the S-IQL-ethyl carbamate is about 1:1 to about 2:1 ml/g.

In one embodiment, the present invention encompasses a process for preparing solifenacin comprising:
a) distilling ethanol with the organic solvent from a reaction mixture comprising (S)-1,2,3,4-tetrahydro-1-phenylisoquinoline-2-carboxylic acid ethyl ester, (R)-1-azabicyclo[2.2.2]octan-3-ol, a base, and an organic solvent; and
b) recycling distilled organic solvent back to the reaction mixture.

In one embodiment, the present invention encompasses a process for preparing a solifenacin salt by converting the obtained solifenacin to a solifenacin salt.

In one embodiment, the present invention encompasses a process for reducing solifenacin diastereomeric and enantiomeric impurities in solifenacin succinate comprising slurrying or crystallizing solifenacin succinate in a mixture of toluene and acetone.

### DETAILED DESCRIPTION OF THE INVENTION

As used herein, the term “room temperature” refers to a temperature of about 15° C. to about 30° C.

As used herein, the term “vacuum” refers to a pressure of about to 2 mmHg to about 100 mmHg.

As used herein, when referring to volume of solvent, the term “constant” means that the volume change is not more than about 10%.

As used herein, when referring to solvent recycling or liquid-liquid extraction, the term “continuous” means the process is uninterrupted in time.

As used herein, the term “SLF” refers to solifenacin and the term “SLF-Suc” refers to solifenacin succinate.

As used herein, the term “solifenacin-SS isomer,” “SLF-SS isomer,” “SLF-SS,” and “solifenacin-SS diastereomer” refers to (3S)-1-azabicyclo[2.2.2]oct-3-yl-(1S)-1-phenyl-3,4-dihydroisoquinoline-2-(1H)-carboxylate and the salts thereof, the term “solifenacin-RR isomer”, “SLF-RR isomer,” “SLF-RR,” and “solifenacin-RR diastereomer” refe to (3R)-1-azabicyclo[2.2.2]oct-3-yl-(1R)-1-phenyl-3,4-dihydroisoquinoline-2-(1H)-carboxylate and the salts thereof, and the term “solifenacin-RS isomer,” “SLF-RS isomer,” “SLF-RS,” and “solifenacin-RS enantiomer” refer to (3S)-1-azabicyclo[2.2.2]oct-3-yl-(1R)-1-phenyl-3,4-dihydroisoquinoline-2-(1H)-carboxylate and the salts thereof.

As used herein, the term “solifenacin diastereomeric and enantiomeric impurities” refer to solifenacin-SS isomer, solifenacin-RR isomer, and solifenacin-RS isomer.

As used herein, the term “IQL” refers to 1,2,3,4-tetrahydro-1-phenylisoquinoline.

As used herein, the term “IQL carbamate” refers to 1,2,3,4-tetrahydro-1-phenylisoquinoline-2-carboxylic acid ethyl ester.

As used herein, the term “S-IQL-ethyl carbamate” refers to (S)-1,2,3,4-tetrahydro-1-phenylisoquinoline-2-carboxylic acid ethyl ester.

As used herein, the term “QNC” refers to 3-quinuclidinol or (R)-1-azabicyclo[2.2.2]octan-3-ol.

As used herein, the term “Me” refers to methyl group, the term “Et” refers to ethyl group, the term “i-Pr” refers to iso-propyl group, and the term “Bu” refers to butyl group.

As used herein, the term “MEK” refers to methylethylketone, the term “MIBK” refers to methyl isobutylketone, the term “MTBE” refers to methyl tert-butyl ether, the term “MeOAc” refers to methyl acetate, the term “EtOAc” refers to ethyl acetate, the term “EtOH” refers to ethanol, the term “EPA” refers to isopropyl alcohol, the term “n-BuOH” refers to n-butanol, the term “DCM” refers to dichloromethane, the term “DMF” refers to N,N-dimethylformamide, the term “DMSO” refers to dimethyl sulfoxide, the term “DMA” refers to dimethylacetamide, and the term “DMC” refers to dimethyl carbonate.

As used herein, the term “RRT” refers to relative retention time, or the ratio between the net retention time of a compound and that of solifenacin succinate under the conditions set forth herein. For example, as used herein, the term “RRT 0.75” refers to a compound with HPLC RRT of about 0.75; the term “RRT 1.06” refers to a compound with HPLC RRT of about 1.06; the term “RRT 1.23” refers to a compound with HPLC RRT of about 1.23. The retention time is measured by HPLC under the following conditions or an equivalent thereof:

Column: Hypersil™ GOLD 250×4.6 mm PN: 25003-254630

Buffer preparation: NaClO₄ (0.01 M), pH 3.0.

Mobile phase:
A: 85% buffer:15% acetonitrile
B: 30% buffer:70% acetonitrile

UV: 210 nm

Column temperature: 25° C.

Flow: 1 mL/min

Volume: 10 μL

Sample preparation: 0.5 mg/mL in mobile phase

As used herein, the term “substantially pure” refers to a the property of having less than about 0.20% of any single chemical impurity as measured by area under HPLC peak relative to the total area under all peaks.

In one embodiment, the present invention encompasses solifenacin succinate having less than about 0.20% of any single impurity as measured by area under HPLC peaks. Preferably, the solifenacin succinate has less than about 0.15% of any single chemical impurity as measured by area under HPLC peaks.

The impurities include, but are not limited to, RRT 0.75, RRT 1.06, and RRT 1.23. Preferably, RRT 0.75 is in an amount of less than about 0.07%, less than about 0.06%, less than about 0.05%, less than about 0.04%, or below detection limit, as measured by area under HPLC peaks. Preferably, RRT 1.06 is in an amount of less than about 0.20%, less than about 0.15%, less than about 0.10% less than about 0.07%, less than about 0.05%, less than about 0.04%, or below detection limit, as measured by area under HPLC peaks. Preferably, RRT 1.23 is in an amount of less than about 0.13%, less than about 0.10%, less than about 0.08%, less than about 0.05%, less than about 0.04%, or below detection limit, as measured by area under HPLC peaks.

Preferably, the chemical purity of the solifenacin succinate is about 99% or more, more preferably about 99.5% or more, more preferably about 99.8% or more, more preferably about 99.9% or more, as measured by area under HPLC peaks.

In one embodiment, the present invention encompasses a process for preparing solifenacin comprising:
a) distilling ethanol with the organic solvent from a reaction mixture comprising (S)-1,2,3,4-tetrahydro-1-phenylisoquinoline-2-carboxylic acid ethyl ester, (R)-1-azabicyclo[2.2.2]octan-3-ol, a base, and an organic solvent; and
b) recycling distilled organic solvent back to the reaction mixture.

S-IQL-ethyl carbamate can be prepared, for example, according to the methods described in Mealy. QNC is available commercially, for example, from OlainFarm.

Ethanol is released during the reaction between S-IQL-ethyl carbamate and QNC. Removing the ethanol from the reaction mixture is preferred because it shifts the reaction equilibrium towards the product. Ethanol can be removed by distillation, preferably by solvent-ethanol mixture co-distillation. Preferably, the ratio between the organic solvent and the S-IQL-ethyl carbamate is about 1:1 to about 4:1 ml/g, more preferably from about 1:1 to about 2:1 ml/g or from about 1:1 to about 1.5:1 ml/g. Compared to the processes disclosed in the EP '304 and EP '965 publications, the processes of the present invention use a smaller amount of solvent. The solvent is recycled during the distillation, allowing for better control of the distillation, and thus reducing the formation of impurities. When applied to an industrial scale production, the processes of the present invention are advantageous in one or more of the following aspects: a) control of the distillation process; b) product quality, including, for example, chemical purity and optical purity; c) economical considerations (for example, less solvent is used); and d) environmental considerations.

Optionally, the distilling and recycling steps take place in an apparatus comprising:
(a) a reaction vessel, wherein the reaction mixture is kept.
(b) a condenser connected directly or indirectly to the reaction vessel, wherein the distilled ethanol and organic solvent are condensed;
(c) a distilling trap connected to the condenser, wherein the condensed ethanol and organic solvent is collected;
(d) a means for connecting the distilling trap to the reaction vessel, through which the organic solvent is recycled back to the reaction vessel. Optionally, the apparatus further comprises: (e) a means to remove liquid from the distilling trap.

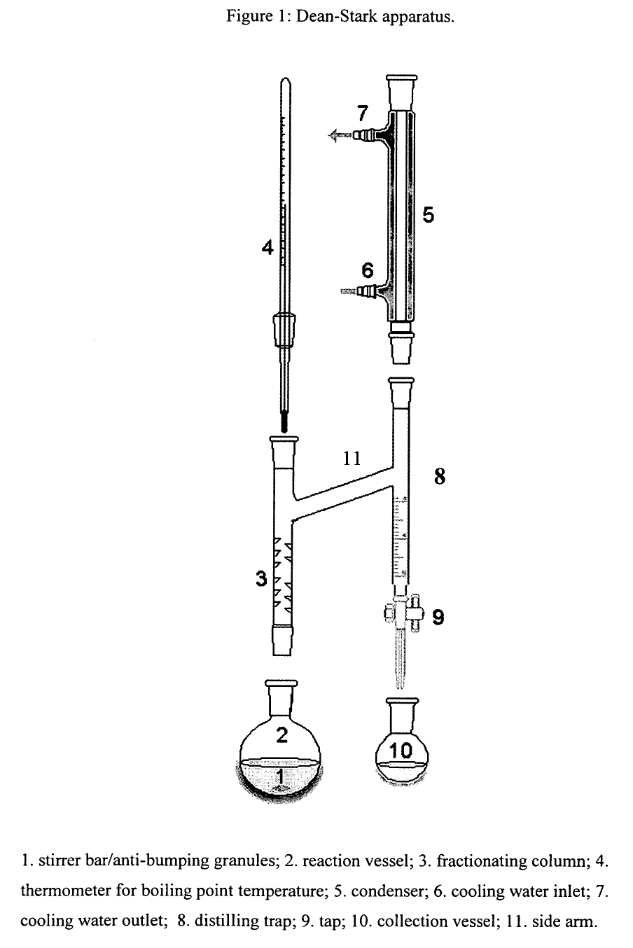

Preferably, the distilling and recycling steps take place in a Dean-Stark apparatus or an equivalent thereof. An example of a Dean-Stark apparatus is shown in FIG. 1. In one embodiment, during the reaction in reaction vessel 2, vapor containing the organic solvent and ethanol is distilled out of the reaction vessel up into the condenser 5, and the condensed organic solvent and ethanol drips into the distilling trap 8. Here, immiscible liquids separate into layers. The liquid in the top layer can flow back to the reaction vessel through side arm 11, while the bottom layer remains in the trap and can be removed through the tap 9 as needed.

Preferably, the volume of the organic solvent in the reaction mixture is kept constant during the distillation. Optionally, the volume change is not more than 5%. Optionally, the recycling of the organic solvent is continuous. If the distilling trap of the apparatus is pre-filled with the organic solvent and optionally a second solvent, then the recycling of the organic solvent starts when the distillation starts, and the volume of the organic solvent in the reaction mixture may be kept constant from the beginning of the distillation. If the distilling trap is not pre-filled with the organic solvent and optionally a second solvent, then the recycling of the organic solvent starts when the distilling trap is filled with distilled organic solvent and optionally a second solvent, and the volume of the organic solvent in the reaction mixture may be kept constant after the recycling starts.

After a mixture of ethanol and the organic solvent is removed from the reaction mixture through distillation, preferably, the ethanol is separated from the organic solvent. Preferably, the ethanol is extracted from the organic solvent by a second solvent that is miscible with ethanol but immiscible with the organic solvent. Preferably, the second solvent is water. Preferably, the extraction is continuous. Preferably, the extraction takes place in the distilling trap of the apparatus. The second solvent is added to the distilling trap prior to or during the distillation to form a second solvent phase. Optionally, a first solvent phase is pre-formed by adding the organic solvent to the distilling trap or formed by the organic solvent distilled from the reaction mixture. The organic solvent, preferably condensed, enters the distilling trap and forms a first solvent layer or remains in the existing first solvent layer, while the ethanol, preferably condensed, is extracted into the second solvent layer. The organic solvent in the first solvent layer is recycled back into the reaction mixture.

Optionally, ethanol is removed from the system. Preferably, ethanol is removed in an mixture of ethanol and the second solvent. Optionally, ethanol is removed through a tap at the bottom of the distilling trap. Preferably, the second solvent is replenished after the removal of the mixture.

As stated above, the ratio between the organic solvent and the S-IQL-ethyl carbamate is preferably about 1:1 to about 4:1 ml/g, more preferably from about 1:1 to about 2:1 ml/g or from about 1:1 to about 1.5:1 ml/g. Preferably, the organic solvent satisfies at least one of the following: (1) has a higher boiling point than ethanol; (2) is able to form an azeotrope with ethanol. Preferably, the organic solvent does not react with the S-IQL-ethyl carbamate or QNC. Optionally, the organic solvent is hydrophobic. Preferably, the organic solvent is hydrophobic when the second solvent is water. Preferably, the organic solvent comprises cyclohexane or an aromatic hydrocarbon. Preferably, the aromatic hydrocarbon is xylene and toluene. More preferably, the aromatic hydrocarbon is toluene. Optionally, the organic solvent further comprises a polar aprotic solvent. Preferably, the polar aprotic solvent is selected from the group consisting of DMF, DMSO, and DMA. More preferably, the polar aprotic solvent is DMF. Preferably, the ratio between the polar aprotic solvent and the S-IQL-ethyl carbamate is about 0.03:1 to about 0.1:1 ml/g. Preferably, the organic solvent is toluene or a mixture of toluene and DMF. Optionally, the S-IQL-ethyl carbamate and QNC are combined in the presence of the organic solvent.

Preferably, the base is selected from the group consisting of alkali metal hydrides, alkali metal amides, and metal alkoxides. Non-limiting examples of alkali metal hydrides include NaH and KH. Non-limiting examples of alkali metal amides include NaNH₂ and KNH₂. Non-limiting examples of metal alkoxides include NaOMe, NaOEt, NaOtBu, KOMe, KOEt, NaOi—Pr, and KOtBu. More preferably, the base is NaH. Preferably, the molar ratio between the base and the S-IQL-ethyl carbamate is about 0.15:1 to about 0.4:1.

Preferably, the reaction mixture is heated to a temperature of about reflux. Preferably, the reaction mixture is refluxed for sufficient time to obtain solifenacin. Preferably, the reaction mixture is refluxed for about 3 to about 8 hours, more preferably for about 4 to about 6 hours.

The obtained solifenacin is preferably recovered, for example, by one or more of the following steps: cooling the mixture after distillation, dilution, washing, and evaporation.

Preferably, the cooling is to a temperature of about room temperature, more preferably about 20° C. to about 25° C.

Preferably, after the distillation, the reaction mixture is diluted with a third organic solvent. Preferably, the third organic solvent added is water immiscible.

Preferably, the third organic solvent comprises a solvent selected from the group consisting of toluene, DCM, EtOAc, and MTBE. More preferably, the solvent is toluene.

Optionally, the diluted or undiluted reaction mixture is washed with water or an aqueous solution of a base. Preferably, the washing is after the dilution. Optionally, the base is an inorganic base such as Na₂CO₃, K₂CO₃, KHCO₃, and NaHCO₃. Optionally, the washing is repeated.

Preferably, the organic solvent in the reaction mixture is evaporated.

Optionally, the obtained solifenacin may be recovered according to the '965 publication by: extracting solifenacin from organic phase with acidic water; adding a base; extracting the solifenacin with an organic solvent; and distilling the organic solvent. Optionally, the acidic water comprises HCl or H₂SO₄. Optionally, the acidic water has a pH of about 4. Optionally, the base is an inorganic base such as Na₂CO₃, K₂CO₃, KHCO₃, and NaHCO₃. Optionally, the organic solvent is toluene, DCM, EtOAc, or MTBE.

Preferably, the solifenacin obtained is substantially pure. Preferably, the solifenacin obtained has a chemical purity of about 95% or more, more preferably about 99.4% or more. Preferably, the solifenacin obtained has about 3% or less solifenacin diastereomeric and enantiomeric impurities as measured by area under HPLC peaks.

Optionally, the obtained solifenacin can be converted to solifenacin salt by, for example, heating the solifenacin-containing organic phase and adding an acid. Preferably, the solifenacin salt is selected from the group consisting of: solifenacin succinate, solifenacin oxalate, and solifenacin hydrochloride. More preferably, the solifenacin salt is solifenacin succinate. Preferably, the heating of the organic phase is to a temperature of about 45° C. to about reflux temperature, more preferably to about 50° C. Preferably, the acid added is selected from the group consisting of succinic acid, oxalic acid, and hydrochloride, more preferably succinic acid. Preferably, the molar amount of the acid added is from about 1 to about 1.1 relative to the molar amount of the solifenacin. Optionally, seeding is done before the succinic acid addition at 45° C.

Optionally, the obtained solifenacin can be converted to solifenacin salt and recovered by, for example, mixing it with an organic solvent and an acid. Optionally, the mixture is heated, preferably to a temperature of about 50° C. Preferably, the organic solvent is selected from the group consisting of toluene, acetone, methylethylketone, methyl isobutylketone, methyl acetate, ethanol, isopropyl alcohol, n-butanol, dimethyl carbonate, and mixtures thereof. Preferably, the solvent is toluene, acetone, or a mixture of toluene and acetone, more preferably a mixture of toluene and acetone. Preferably, the acid is selected from the group consisting of succinic acid, oxalic acid, and hydrochloride, more preferably succinic acid. Preferably, the molar amount of the acid added is from about 1 to about 1.1 relative to the molar amount of the solifenacin.

Solifenacin may be converted to solifenacin succinate by reacting with succinic acid, for example, according to the methods disclosed in WO 2005/087231, copending U.S. patent application Ser. No. 11/645,021, published as US 20070173528, and copending U.S. patent application Ser. No. 11/881,161 (“the '161 application”), published as US 20080114028, all of which are incorporated herein by reference.

The obtained solifenacin salt can be recovered, for example, through crystallization. Optionally, the crystallization is done by one or more of the following steps: cooling, seeding, obtaining a slurry, stirring, and isolating the solifenacin salt. Optionally, the slurry is stirred. Preferably, the stirring is done for about 0 to about 20 hours, more preferably for about 16 hours. Preferably, the stirring is done at a temperature of about 50° C. Optionally, the slurry is cooled. Preferably, the cooling is to a temperature of about 0° C. to about 30° C., more preferably to about room temperature or about 20° C. to about 25° C. Preferably, the cooling is done while stirring. Preferably, the stirring is done for about 2 to about 24 hours, more preferably for about 4 hours. The '161 application describes crystallization of solifenacin succinate.

Optionally, the solifenacin salt is further isolated by one or more of the following steps: vacuum filtration, washing with an organic solvent, and drying in an oven. The drying temperature is preferably from about 40° C. to about 60° C., more preferable about 55° C. Preferably, the drying is for about 6 to about 48 hrs, more preferably for overnight to about 24 hours. Optionally, the drying is done under vacuum, preferably at a pressure of about 2 to about 60 mmHg. WO 2008/013851 describes such recovery.

Preferably, the solifenacin salt obtained is substantially pure. Preferably, the obtained solifenacin has less than about 0.20% of any single chemical impurity as measured by area under HPLC peaks. Preferably, the obtained solifenacin has a chemical purity of about 99% or more, about 99.5% or more, about 99.8% or more, or about 99.9% or more. The purity of solifenacin salt, specifically solifenacin succinate, obtained by method in accordance with the invention is exemplified in Examples 12 and 13.

Preferably, the obtained solifenacin succinate has RRT 0.75 in an amount of less than about 0.07%, less than about 0.06%, less than about 0.05%, less than about 0.04%, or below detection limit, as measured by area under HPLC peaks. Preferably, the obtained solifenacin succinate has RRT 1.06 in an amount of less than about 0.20%, less than about 0.15%, less than about 0.10% less than about 0.07%, less than about 0.05%, less than about 0.04%, or below detection limit, as measured by area under HPLC peaks. Preferably, the obtained solifenacin succinate has RRT 1.23 in an amount of less than about 0.13%, less than about 0.10%, less than about 0.08%, less than about 0.05%, less than about 0.04%, or below detection limit, as measured by area under HPLC peaks.

Preferably, the obtained solifenacin succinate has total diastereomeric and enantiomeric impurity level of about 0.50% or less, about 0.40% or less, about 0.30% or less, about 0.20% or less, about 0.10% or less, about 0.05% or less, about 0.04% or less, or below detection limit.

In one embodiment, the present invention encompasses a process for reducing solifenacin diastereomeric and enantiomeric impurities in solifenacin succinate comprising slurrying or crystallizing solifenacin succinate in a mixture of toluene and acetone.

The ratio of toluene to solifenacin succinate is preferably about 1 ml/g to about 3.5 ml/g, more preferably about 1 ml/g. The ratio of acetone to solifenacin succinate is preferably about 3.5 ml/g to about 15 ml/g, more preferably about 15 ml/g.

The slurry is preferably heated to about 40° C. to reflux temperature, more preferably to reflux temperature. The heating is preferably maintained for about 20 minutes to about 3 hours, more preferably about 30 to about 80 minutes. Optionally, the slurry is cooled to about 9° C. to about 25° C., more preferably to about 9 to about 14° C. The cooling is preferably maintained for about 2 to about 5 hours, more preferably about 2.5 hours.

Optionally, the slurry is filtered. Optionally, the filter cake obtained is washed with at least one of toluene and acetone. Optionally, the filter cake is dried, preferably at a temperature of about 40° C. to about 55° C., preferably under vacuum, more preferably a pressure of about to 2 to about 60 mmHg.

Preferably, the solifenacin diastereomeric and enantiomeric impurity level in the solifenacin succinate is reduced by about 85% or more. Preferably, the solifenacin succinate obtained has about 0.03% or less, or below detection limit, any of the solifenacin diastereomeric and enantiomeric impurities as measured by area under HPLC peaks.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Analytical Methods

Unless otherwise specified, the optical purity is determined by the following method:

Column: Chiralpak® AD-H 250×4.6 mm

Mobile phase: hexane:ethanol:diethylamine (800:200:1)

UV: 220 nm

Column temperature: 20° C.

Flow: 1 mL/min

Volume: 10 μL

Sample preparation: 0.3 mg/mL in a mixture of hexane/ethanol (1:1)

Retention time for solifenacin succinate: about 25 min.

Unless otherwise specified, the chemical purity is determined by the following method:

Column: Hypersil™ GOLD 250×4.6 mm PN: 25003-254630

Buffer preparation: NaClO₄ (0.01M), pH 3.0.

Mobile phase:

A: 85% buffer:15% acetonitrile

B: 30% buffer:70% acetonitrile

UV: 210 nm

Column temperature: 25° C.

Flow: 1 mL/min

Volume: 10 μL

Sample preparation: 0.5 mg/mL in mobile phase

Retention time for solifenacin succinate: about 11 min.

### Example 1

A 100 ml round bottom flask equipped with mechanical stirrer, thermometer and Dean-stark condenser (filled with some water), was loaded with S-IQL-ethyl carbamate (25 g), toluene (25 ml), QNC (19.78 g), and NaH (60% in mineral oil, 1.6 g). The mixture was heated to reflux and stirred for 3 hours. The mixture was cooled to room temperature and diluted with toluene to 10 ml/g of S-IQL-ethyl carbamate and washed twice with water (100 ml followed by 70 ml). The organic phase was heated to 50° C., and succinic acid (10.4 g) was added. After 10 minutes, a slurry was obtained. The mixture was stirred at 50° C. for 16 hours, and then cooled to RT and stirred for 4 hours. SLF succinate crude was isolated by vacuum filtration, washed with toluene (2×40 ml), and dried in a vacuum oven at 55° C. for 24 hours. (Yield: 31 g; chemical purity: 98.70%; optical purity: 99.60%)

TABLE 1

| | | 0.75 | 1.0 | | | 0.83 |
|---|---|---|---|---|---|---|
| | | IQL | SLF-SUC | 1.0 | 1.0 | IQL Carbamate |
| | Retention | 4.33 | 7.91 | 8.74 | 10.28 | |
| | Time | | | | | |
| Example 1 | RRT | 0.55 | 1.00 | 1.11 | 1.30 | 0.00 |
| | % on Area | 0.62 | 98.70 | 0.10 | 0.14 | 0.00 |

### Example 2

A 100 ml round bottom flask equipped with mechanical stirrer, thermometer and Dean-Stark condenser (filled with some water), was loaded with S-IQL-ethyl carbamate (27 g), toluene (2 ml), DMF (1.35 ml), QNC (18.4 g), and NaH (60% in mineral oil, 1.15 g). The mixture was heated to reflux and stirred for 4.5 hours while being monitored by HPLC. The mixture was cooled to room temperature. Half of the reaction mixture was diluted with toluene (54 ml) and washed with water (67 ml). The organic phase was evaporated, and toluene (14.6 ml) and acetone (131 ml) were added to the evaporated residue. Succinic acid (4.74 g) was added and the solution was heated to 50° C. After 20 minutes, the mixture was cooled to room temperature and stirred for 5 hours. Seeding was implemented and the slurry was stirred over night. SLF succinate was isolated by vacuum filtration, washed with acetone (2×25 ml), and dried in a vacuum oven at 55° C. over night. (Yield: 13.6 g)

### Example 3

A 100 ml round bottom flask equipped with mechanical stirrer, thermometer and Dean-stark condenser (filled with some water), was loaded with S-IQL-ethyl carbamate (25 g), toluene (25 ml), DMF (1.25 ml), QNC (13.5 g), and stirred at room temperature for 10 minutes. NaH (60% in mineral oil, 0.53 g) was then added. The mixture was heated to reflux and stirred for 6 hours, while being monitored by HPLC. The mixture was cooled to room temperature and washed with water (75 ml) followed by NaHCO₃ solution (2.5%, 75 ml). The organic phase was evaporated to obtain an oily SLF base. Table 2 summarizes the optical purity results and the chemical purity results of this Example and an example prepared according to the EP '965 publication (corresponds to WO 2005/075474).

TABLE 2

| | | Reference |
|---|---|---|
| | | Example 1 |
| | Example 3 | Production of SLF |
| | (% area by HPLC¹) | EP 1,714,965² |
| Solifenacin-RR diastereomer | 2.26 | 4.51 |
| Solifenacin-SS diastereomer | 0.69 | 2.33 |
| Solifenacin-RS enantiomer | Less than 0.03 | 0.14 |
| IQL | 0.25 | 0.32 |
| IQL carbamate | 2.28 | 1.07 |

¹Optical purity analyzed by using the Hypersil ™ method described above; chemical purity analyzed by using the Chiralpak ® method described above.
²The values refer to the content ratio of the specific impurity with regard to solifenacin, wherein solifenacin is defined as 100%.

### Example 4

SLF succinate crude (3 g) was mixed with acetone (30 ml), and heated to reflux. Acetone (40 ml) was added gradually for dissolution. The solution was cooled to room temperature and stirred for 4 hours. The product was isolated by vacuum filtration, washed with acetone (15 ml), and dried in vacuum oven at 55° C. overnight to obtain crystalline solifenacin succinate. (Yield: 2.06 g; chemical purity: 99.74%; optical purity: 99.91%)

### Example 5

SLF succinate crude (3 g) was mixed with MEK (30 ml), and heated to reflux for dissolution. The solution was then cooled to room temperature and stirred for 4.5 hours. The product was isolated by vacuum filtration, washed with MEK (10 ml), and dried in vacuum oven at 55° C. overnight to obtain crystalline solifenacin succinate. (Yield: 2.65 g; chemical purity: 99.82%; optical purity: 99.96%)

### Example 6

SLF succinate crude (3 g) was mixed with MIBK (30 ml) and heated to reflux. MIBK (60 ml) was added gradually for dissolution. The solution was then cooled to room temperature and stirred at room temperature for 2 hours and at 0° C. for 0.75 hour. The product was isolated by vacuum filtration, washed with MIBK, and dried in vacuum oven at 55° C. overnight to obtain crystalline solifenacin succinate. (Yield: 2.35 g; chemical purity: 99.43%; optical purity: 99.93%)

### Example 7

SLF succinate crude (3 g) was mixed with MeOAc (30 ml) and heated to reflux. MeOAc (50 ml) was added gradually for partial dissolution. The mixture was then cooled to room temperature and stirred at room temperature for 2 hours and at 0° C. for 0.75 hour. The product was isolated by vacuum filtration, washed with MeOAc, and dried in vacuum oven at 55° C. overnight to obtain crystalline solifenacin succinate pure (Yield: 2.44 g; chemical purity: 99.78%; optical purity: 99.50%)

TABLE 3

| | | 0.75 | 1.0 | | |
|---|---|---|---|---|---|
| | | Sample Name | | | |
| | | IQL | SLF-SUC | 1.0 | 1.0 |
| Example 4 | Retention Time | | 7.906 | 8.733 | 13.51 |
| | RRT | 0.00 | 1.00 | 1.10 | 1.71 |
| | % on Area | 0.00 | 99.74 | 0.04 | 0.04 |
| Example 5 | Retention Time | | 7.90 | 8.74 | |
| | RRT | 0.00 | 1.00 | 1.11 | 0.00 |
| | % on Area | 0.03 | 99.82 | 0.04 | 0.00 |
| Example 6 | Retention Time | 4.34 | 7.92 | 8.74 | |
| | RRT | 0.55 | 1.00 | 1.10 | 0.00 |
| | % on Area | 0.05 | 99.43 | 0.18 | 0.00 |
| Example 7 | Retention Time | | 7.90 | 8.73 | |
| | RRT | 0.00 | 1.00 | 1.10 | 0.00 |
| | % on Area | 0.00 | 99.78 | 0.07 | 0.00 |

### Example 8

SLF succinate crude (3 g) was mixed with EtOH (10 ml) and heated to reflux for dissolution. The solution was then cooled to room temperature and stirred at room temperature for 1.75 hours and at 0° C. for 0.33 hour. The product was isolated by vacuum filtration, washed with EtOH (10 ml), and dried in vacuum oven at 50° C. overnight to obtain crystalline solifenacin succinate. (Yield: 2.38 g; chemical purity: 99.68%; optical purity: 99.96%)

### Example 9

SLF succinate crude (3 g) was mixed with IPA (10 ml) and heated to reflux for dissolution. The solution was then cooled to room temperature and stirred at room temperature for 1.75 hours and at 0° C. for 0.5 hour. The product was isolated by vacuum filtration, washed with IPA, and dried in vacuum oven at 50° C. overnight to obtain crystalline solifenacin succinate. (Yield: 2.51 g; chemical purity: 99.46%; optical purity: 99.77%)

### Example 10

SLF succinate crude (3 g) was mixed with n-BuOH (10 ml) and heated to reflux. n-BuOH (10 ml) was added for dissolution. The solution was then cooled to room temperature and stirred at room temperature for 1.25 hours and at 0° C. for 0.5 hour. The product was isolated by vacuum filtration, washed with n-BuOH, and dried in vacuum oven at 50° C. overnight to obtain crystalline solifenacin succinate. (Yield: 2.26 g; chemical purity: 99.70%; optical purity: 99.82%)

TABLE 4

| | | Sample Name | | | |
|---|---|---|---|---|---|
| | | IQL | SLF-SUC | | |
| Example 8 | Retention Time | 4.282 | 7.755 | 10.09 | |
| | RRT | 0.55 | 1.00 | 1.30 | 0.00 |
| | % on Area | 0.24 | 99.68 | 0.06 | 0.00 |
| Example 9 | Retention Time | 4.29 | 7.74 | 10.07 | |
| | RRT | 0.55 | 1.00 | 1.30 | 0.00 |
| | % on Area | 0.32 | 99.46 | 0.08 | 0.00 |
| Example 10 | Retention Time | 4.28 | 7.72 | 10.06 | |
| | RRT | 0.55 | 1.00 | 1.30 | 0.00 |
| | % on Area | 0.19 | 99.70 | 0.04 | 0.00 |

TABLE 5

| | SLF-RS | SLF-SS | SLF-RR | |
|---|---|---|---|---|
| | 0.6 | 0.7 | 0.8 | SLF |
| Example 1 | — | 0.33 | 0.07 | 99.60 |
| Example 4 | | 0.04 | 0.05 | 99.91 |
| Example 5 | | 0.04 | | 99.96 |
| Example 6 | | 0.06 | | 99.93 |
| Example 7 | | 0.04 | | 99.50 |
| Example 8 | | 0.04 | | 99.96 |
| Example 9 | — | 0.19 | 0.04 | 99.77 |
| Example 10 | — | 0.15 | 0.03 | 99.82 |

### Example 11

SLF succinate crude (2.6 g) was mixed with DMC (26 ml) and heated to reflux. DMC (10 ml) was added for dissolution, but a clear solution was not observed. The mixture was then cooled to room temperature and stirred for about 3 hours. The product was isolated by vacuum filtration, washed with DMC, and dried in vacuum oven at 55° C. overnight to obtain solifenacin succinate pure (Yield: 1.25 g; chemical purity: 82.29%)

TABLE 6

| | | | | | | IQL |
|---|---|---|---|---|---|---|
| | | IQL | SLF-SUC | | | Carbamate |
| Example 11 | Retention | 5.26 | 8.80 | 9.22 | 10.77 | |
| | Time | | | | | |
| | RRT | 0.60 | 1.00 | 1.05 | 1.22 | 0.00 |
| | % on | 0.28 | 82.29 | 7.40 | 0.08 | 0.00 |
| | Area | | | | | |

### Example 12

A 100 ml round bottom flask equipped with a mechanical stirrer, a thermometer, and a Dean-Stark condenser (filled with some water) was loaded with a solution of S-IQL-ethyl carbamate (31.78 g) in toluene (1 ml/g of S-IQL-ethyl carbamate), DMF (1.58 ml), QNC (18.45 g), and NaH (60%, 0.09 g) in its original bag (SECUBAG, styrene-butadiene-styrene copolymer, 0.15 g). The mixture was heated to reflux and stirred for 5.5 hours. The reaction was monitored by HPLC, and the water in the Dean-Stark condenser was replenished several times during the reaction process. The resulting mixture was cooled to room temperature, diluted with toluene (65 ml), and washed with water (97 ml). The organic phase was washed with 1.5% Na₂CO₃ solution (97 ml) and evaporated. Toluene (43 ml) and acetone (646 ml) were added to the evaporated residue. Succinic acid (13.33 g) was then added. The resulting solution was heated to 50° C. and stirred for 30 minutes (precipitation occurred after a few minutes). The resulting mixture was cooled to room temperature and stirred overnight. The SLF succinate was isolated by vacuum filtration and dried in a vacuum oven at 55° C. over a weekend. (yield: 41 g, 75.6%) The chemical purity results of the Example are listed in Table 7.

### Example 13

A 100 ml round bottom flask equipped with a mechanical stirrer, a thermometer, and a Dean-stark condenser (filled with some water) was loaded with S-IQL-ethyl carbamate (33.6 g), toluene (33.6 ml), DMF (1.68 ml), QNC (19.87 g), NaH (60% in mineral oil, 0.95 g), and the bag (SECUBAG, styrene-butadiene-styrene copolymer, 0.159 g). The mixture was heated to reflux and stirred for 6.5 hours. The reaction was monitored by HPLC, and the water in the Dean-Stark condenser was replenished several times during the reaction process. The resulting mixture was cooled to room temperature, diluted with toluene (67.2 ml), and washed with water (100 ml). The organic phase was then washed with 1.5% Na₂CO₃ solution (100 ml) and evaporated. Toluene (44.3 ml) and acetone (664.5 ml) were added to the evaporated residue. Succinic acid (14 g) was then added. The resulting solution was heated to 50° C. and stirred for 30 minutes (precipitation occurred after a few minutes). The resulting mixture was cooled to room temperature and stirred overnight. The SLF succinate was isolated by vacuum filtration and dried in vacuum oven at 55° C. over a weekend. (yield: 36.9 g, 64.35%) The chemical purity results of the Example are listed in Table 7.

TABLE 7

| | | Sample Name | | | | | |
|---|---|---|---|---|---|---|---|
| | | | | | | | IQL |
| | | IQL | | SLF-SUC | | | Carbamate |
| | Retention | 5.958 | 8.157 | 10.909 | 11.571 | 13.37 | |
| | Time | | | | | | |
| | RRT | 0.55 | 0.75 | 1.00 | 1.06 | 1.23 | 0.00 |
| VESIcare ® 10 mg | Area % by | 0.27 | 0.07 | 98.51 | 0.24 | 0.13 | 0.00 |
| tablet | HPLC | | | | | | |
| VESIcare ® 5 mg | Area % | 0.27 | 0.11 | 98.08 | 0.21 | 0.15 | 0.00 |
| tablet | | | | | | | |
| Example 12 | Area % | 0.03 | 0.00 | 99.79 | 0.00 | 0.00 | 0.00 |
| Example 13 | Area % | 0.02 | 0.00 | 99.86 | 0.00 | 0.00 | 0.00 |

### Example 14

A 100 L reactor was loaded with S-IQL (8.5 kg), toluene (7 ml/g of S-IQL), water (1.6 ml/gram of S-IQL), and Na₂CO₃ (0.6 molar eq. to S-IQL). Ethylchloroformate (4.9 kg) was dripped slowly to the reactor. During feeding the temperature inside the reactor increased from 14.9° C. to 34.8° C. The mixture was stirred at 25° C. for 3 hours. Then the reactor mixture (which contains two phases) was circulated on a GAF filter with filter aid (HYFLO SUPER-CEL, Johns Manville Corp.). The filtrate was separated and concentrated to obtain 41 kg of S-IQL ethyl carbamate solution. The solution contained 11.4 kg of S-IQL ethyl carbamate in 34 L of toluene (3 L/kg of S-IQL-ethyl carbamate).

40 kg of the above solution of S-IQL-ethyl carbamate was transferred to another 100 L reactor. Toluene (0.2 L/kg of S-IQL-ethyl carbamate), QNC (1.4 molar eq. to S-IQL ethyl carbamate), and NaH (0.26 molar eq. to S-IQL ethyl carbamate) in its original bag (SECUBAG) were added. When foaming ended, DMF (0.05 L/kg of S-IQL-ethyl carbamate) was added. Toluene (2.16 L/kg of S-IQL-ethyl carbamate) was distilled out. Then the reaction mixture was distilled through a Dean-Stark condenser filled with 25 L of water and 30 L of toluene.

After 5 hours, the water inside the Dean-Stark condenser was refreshed and the reaction continued for another hour. Toluene (2 L/kg of S-IQL-ethyl carbamate) was added. The organic solution was washed with tap water (3 L/kg of S-IQL-ethyl carbamate) and Na₂CO₃ solution (3 L/kg of S-IQL-ethyl carbamate, 0.5% w/w in tap water, pH=10.5).

The solifenacin base solution was transferred to a 160 L reactor through a 5 μm GAF filter with filter aid (HYFLO SUPER-CEL), and through 1 μm and 0.2 μm filters. Then the solution was concentrated by distillation until no more distillate was obtained.

Afterwards, acetone (9 L/kg of S-IQL-ethyl carbamate) was added. The mixture was heated to 45° C. Seeding was carried out with solifenacin succinate. Then succinic acid (1 molar eq. to S-IQL ethyl carbamate) was fed to the reactor. Precipitation began after 12 min at 44.5° C.

The reactor was kept at around 44° C. for 72 min, cooled to 14.2° C. during 3.6 hours, and stirred at around 13° C. for 2.5 hours. Half of the mixture was filtered. The filtrate was slurried with 4.4 L/kg of S-IQL-ethyl carbamate of toluene for 45 min and then filtered. The second half was filtered after 11 hours and washed with toluene (2.5 L/kg of S-IQL-ethyl carbamate). Each cake was then washed twice with 2 L/kg of S-IQL ethyl-carbamate of acetone.

From each cycle, about 2 kg was taken out of the filter drier to be dried in the fluidized bed drier at 50° C. for 2.5 hours. The rest of each cycle was dried in the filter drier at 50° C. for 4 hours. (Total yield 77%, optical purity 100%)

### Example 15

### Reprocessing of Solifenacin Succinate

Solifenacin succinate (9.7 kg, containing 0.2% of SLF-S.S isomer) was fed to a 400 L reactor. Toluene (1 L/kg of solifenacin succinate) and acetone (15 L/kg of solifenacin succinate) were added. The resulting slurry was heated to reflux (56° C.) for 80 min. Then the slurry was cooled to 13.7° C. during 100 min, and kept at 9.5-13.7° C. for 2.5 hours before filtration.

The slurry containing SLF succinate was filtered. The cake was washed once with toluene (2 L/kg of solifenacin succinate) and than twice with acetone (2 L/kg of solifenacin succinate). After filtration the solifenacin succinate cake was not discharged and was dried at 45° C., 49-55 mmHg, and 13 rpm for 4.3 hrs.

8.6 kg of solifenacin succinate dry was obtained. (Loss on drying=0.2%; chemical purity: 99.98%; SLF-SS isomer less than 0.03%; SLF-RR isomer less than 0.03%)

TABLE 8

| | | | Diastereomeric and | | | | | |
|---|---|---|---|---|---|---|---|---|
| | | Assay | Enantiomeric Purity (%) | | | | | IQL |
| | Step | (%) | SLF-SS | SLF-RR | SLF | S-IQL | SLF | Carbamate |
| Example | SLF Base | N.A. | 0.67 | 0.43 | 99.28 | 0.34 | 97.71 | 1.88 |
| 15 | SLF-Suc | 100.4 | Less than | Less than | 100 | 0.02 | 99.98 | Less than |
| | | | 0.03 | 0.03 | | | | 0.03 |

1. A process for preparing solifenacin comprising:
a) distilling ethanol and the organic solvent from a reaction mixture comprising (S)-1,2,3,4-tetrahydro-1-phenylisoquinoline-2-carboxylic acid ethyl ester, (R)-1-azabicyclo[2.2.2]octan-3-ol, a base, and an organic solvent; and
b) recycling distilled organic solvent back to the reaction mixture.

2. The process of claim 1, wherein the volume of the organic solvent in the reaction mixture is kept constant during the distillation.

3. The process of claim 1, wherein the recycling step is continuous.

4. The process of claim 1, wherein the base is selected from the group consisting of alkali metal hydrides, alkali metal amides, and metal alkoxides.

5. (canceled)

6. The process of claim 1, wherein the molar ratio between the base and the (S)-1,2,3,4-tetrahydro-1-phenylisoquinoline-2-carboxylic acid ethyl ester is about 0.15:1 to about 0.4:1.

7. The process of claim 1, wherein the organic solvent satisfies at least one of the following: (1) has a higher boiling point than ethanol; (2) is able to form an azeotrope with ethanol.

8-14. (canceled)

15. The process of claim 1, wherein the ratio of the organic solvent to (S)-1,2,3,4-tetrahydro-1-phenylisoquinoline-2-carboxylic acid ethyl ester in step a) is about 1:1 to about 4:1 mL/g.

16. (canceled)

17. (canceled)

18. The process of claim 1, wherein the distilled ethanol is separated from the distilled organic solvent.

19. (canceled)

20. (canceled)

21. The process of claim 1, wherein the distilling and recycling steps take place in an apparatus comprising,
(a) a reaction vessel, wherein the reaction mixture is kept.
(b) a condenser connected directly or indirectly to the reaction vessel, wherein the distilled ethanol and organic solvent are condensed;
(c) a distilling trap connected to the condenser, wherein the condensed ethanol and organic solvent is collected;
(d) a means for connecting the distilling trap to the reaction vessel, through which the organic solvent is recycled back to the reaction vessel.

22. (canceled)

23. The process of claim 1, wherein the distilling and recycling steps take place in a Dean-Stark apparatus or an equivalent thereof.

24-29. (canceled)

30. The process of claim 1, wherein the distilling step comprises refluxing the reaction mixture.

31-39. (canceled)

40. The process of claim 1, wherein the solifenacin obtained has a chemical purity of about 95% or more by area under HPLC peaks.

41. (canceled)

42. The process of claim 1, wherein the solifenacin obtained has about 3% or less solifenacin diastereomeric and enantiomeric impurities as measured by area under HPLC peaks.

43. A process for preparing a solifenacin salt, comprising preparing solifenacin according to the process of claim 1 and converting the solifenacin obtained to a solifenacin salt.

44. (canceled)

45. The process of claim 43, wherein the solifenacin salt is solifenacin succinate.

46. The process of claim 45, wherein the solifenacin succinate obtained has less than about 0.20% of any single chemical impurity as measured by area under HPLC peaks.

47. (canceled)

48. A process for reducing solifenacin diastereomeric and enantiomeric impurities in solifenacin succinate comprising slurrying or crystallizing solifenacin succinate in a mixture of toluene and acetone.

49. The process of claim 48, wherein the ratio of toluene to solifenacin succinate is preferably about 1 ml/g to about 3.5 ml/g.

50. The process of claim 48, wherein the ratio of acetone to solifenacin succinate is preferably about 3.5 ml/g to about 15 ml/g.

51. The process of claim 48, wherein the slurry is preferably heated to about 40° C. to reflux temperature.

52. The process of claim 51, wherein the heating is maintained for about 20 minutes to about 3 hours.

53. The process of claim 48, wherein the slurry is cooled to about 9° C. to about 25° C.

54. The process of claim 53, wherein the cooling is maintained for about 2 to about 5 hours.

55. The process of claim 48, wherein the diastereomeric and enantiomeric impurity level in the solifenacin succinate is reduced by about 85% or more.

56. The process of claim 48, wherein the solifenacin succinate obtained has about 0.03% or less any of the solifenacin diastereomeric and enantiomeric impurities as measured by area under HPLC peaks.
